(19) United States
(12) Patent Application Publication
(10) Pub. No.: US 2016/0073922 A1
(43) Pub. Date: Mar. 17, 2016

(54) ROBUST TARGETING OF PHOTOSENSITIVE MOLECULES

(71) Applicant: THE TRUSTEES OF THE UNIVERSITY OF PENNSYLVANIA, Philadelphia, PA (US)

(72) Inventors: Geoffrey Karl Aguirre, Philadelphia, PA (US); David Brainard, Merion Station, PA (US); Manuel Spitschan, Philadelphia, PA (US)

(21) Appl. No.: 14/852,001

(22) Filed: Sep. 11, 2015

Related U.S. Application Data
(60) Provisional application No. 62/049,242, filed on Sep. 11, 2014.

(51) Int. Cl. A61B 5/0484; A61B 5/16; A61B 5/0488; A61B 3/11; A61B 5/00; A61B 5/0496

(52) U.S. Cl. CPC A61B 5/04842; A61B 5/0077; A61B 5/0496; A61B 5/0488; A61B 3/112; A61B 5/4082; A61B 5/165; G01N 2201/062

(57) ABSTRACT

The present disclosure relates to systems and methods that can be used to stimulate and record responses elicited from a naturally-occurring or artificially-introduced light-sensitive molecule. In certain non-limiting embodiments, a system of the presently disclosed subject matter includes (a) a digital spectral integrator, e.g., light source, (b) a detection means and (c) an integration means.

### CROSS-REFERENCE TO RELATED APPLICATIONS

This application claims priority to U.S. Provisional Patent Application Ser. No. 62/049,242, filed Sep. 11, 2014, the contents of which are incorporated by reference herein in their entirety.

### GRANT INFORMATION

This disclosure was made with government support under Grant Numbers 5R01EY020516, 5R01EY010016 and P30EY001583 awarded by National Institutes of Health. The government has certain rights in the invention.

### INTRODUCTION

The present disclosure relates to systems and methods that can be used to stimulate and record responses elicited from naturally-occurring or artificially-introduced light-sensitive molecules.

### BACKGROUND OF THE DISCLOSURE

Rods and cones of the retina are the well known light sensing cells of the eye. Melanopsin-expressing retinal ganglion cells (ipRGCs) have been identified as an additional population of photoreceptor cells that regulate circadian rhythm, painful sensitivity to bright light and the pupil light reflex. There is some early evidence that melanopsin containing cells contribute to human visual perception of light intensity. ipRGCs are of particular interest as their function may be linked to various clinical conditions. Thus, the ability to assess the function of melanopsin can be useful in the diagnosis and treatment of clinical conditions. More generally, there is a need for a technique that can selectively probe the function of the different classes of human photoreceptors in a repeatable manner that is robust to incidental biological variation.

ipRGCs comprise less than 1-3% of total retinal ganglion cells. The function of ipRGCs is difficult to measure because the spectral sensitivity of melanopsin overlaps with the spectral sensitivity of the rods and the cones. Methods to probe the function of melanopsin in humans to date have been based upon measurement of the differential evoked pupil response to a blue or red flash of light (termed the post-illumination pupil response or PIPR), or to modulations of combinations of primary lights (typically 4). These techniques have limitations in their ability to probe responses at different temporal frequencies, in their extensibility to other photoreceptor classes and in their robustness to incidental biological variation (e.g., age and photoreceptor allelic differences) that can influence the measures.

To address this deficiency, the present disclosure provides a rapid, robust and non-invasive measurement system for determining relative sensitivity of the melanopsin photoreceptor system. However, as outlined herein, this system is not limited to use in the contexts of melanopsin or photosensitive retinal cell analysis, but instead can be used to stimulate and record responses elicited from essentially any naturally-occurring or artificially-introduced light-sensitive molecule.

### SUMMARY OF THE DISCLOSURE

In certain embodiments, the present disclosure relates to a system including (a) a digital spectral integrator, e.g., a light source, (b) a detection means and (c) an integration means. In certain embodiments, the light source produces a light of a specified spectral and temporal profile under the control of the integration means, and the detection means detects a response to the light of a specified spectral and temporal profile.

In certain embodiments, the detection means is an infrared camera, electroretinogram, electroencephalogram, functional MRI scanner, electromyogram of periorbital muscles or recording of a subject's behavioral response. In certain embodiments, the integration means is a software program. In certain embodiments, the software program can interpret the response to the light of a specified spectral and temporal profile, e.g., pupil movement, electrical response of the retina, neural activity of the brain and/or perceptual report from the subject. In certain embodiments, the light of a specified spectral and temporal profile is spatially uniform.

In certain embodiments, the present disclosure relates to a method of selectively stimulating photosensitive molecules in a manner that is robust to biological variation in age, peak spectral sensitivity of photoreceptors, spatial location on the retina and the presence of retinal blood vessels.

In certain embodiments, the present disclosure relates to a method of detecting a response or producing a biological effect associated with selectively stimulating a photosensitive molecule including (a) contacting the photosensitive molecule with a light of a specified spectral and temporal profile, (b) robustly silencing responses from non-targeted photosensitive molecules despite biological variation in the spectral sensitivity of the photosensitive molecules and (c) detecting the response or biological effect associated with selective stimulation of the photosensitive molecule.

In certain embodiments, the light of a specified spectral and temporal profile has a carrier frequency, e.g., sinusoidal carrier frequency. For example, and not by way of limitation, the carrier frequency is of about 0.01 Hz to about 128 Hz.

In certain embodiments, the light of a specified spectral and temporal profile includes a lower envelope frequency and a higher carrier frequency. For example, and not by way of limitation, the higher carrier frequency is of about 4 Hz to about 128 Hz. In certain embodiments, the lower envelope frequency is of about 0.01 Hz to 1 Hz.

In certain embodiments, the photosensitive molecule has been artificially introduced into neural tissue, e.g., the central nervous system or peripheral nervous system. In certain embodiments, the neural tissue is the retina. In certain embodiments, the photosensitive molecule is a photosensitive protein. In certain embodiments, the photosensitive protein is present in a photosensitive cell. In certain embodiments, the photosensitive cell is a retinal cell. In certain embodiments, the retinal cell is a rod, cone or a melanopsin-expressing retinal ganglion cell. In certain embodiments, the retinal cell is a melanopsin-expressing retinal ganglion cell.

In certain embodiments, the present disclosure relates to a method of detecting an impairment in a temporal sensitivity of a photosensitive cell in a subject that includes (a) contacting the photosensitive cell with a light of a spectral and temporal profile and (b) detecting the response associated with selective stimulation of the photosensitive molecule, wherein the detection of an impairment in the temporal sensitivity of a photosensitive cell is indicative that the subject has an ophthalmologic, neurological or psychiatric disorder. In certain embodiments, the retinal cell is a rod, cone or a melanopsin-expressing retinal ganglion cell. In certain embodiments, the retinal cell is a melanopsin-expressing retinal ganglion cell.

In certain embodiments, the psychiatric disorder can include seasonal affective disorder and other mood disorders. In certain embodiments, the neurological disorder can include migraines, photophobia, traumatic brain injury and neurodegenerative disorders involving the brainstem, such as, but not limited to, Progressive Supra-nuclear Palsy. In certain embodiments, the ophthalmologic disorder can include disorders of retinal function such as, but not limited to, hereditary retinopathies including Leber's Congenital Amaurosis and retinitis pigmentosa, and acquired retinopathies such as, but not limited to, diabetic retinopathy and glaucoma.

### BRIEF DESCRIPTION OF THE FIGURES

FIG. 1A-B depicts (a) the four known light sensitive cells of the eye under daylight conditions: the three cones (S, M and L) and melanopsin-containing retinal ganglion cells (ipRGCs). Rods are also present in the eye and operate under dim light conditions. Recent work suggests that melanopsin ipRGCs contribute to the pupillary response, circadian rhythm and photophobia. These functions have many connections to clinical disease. (b) The spectral sensitivity of S, M and L-cones and the extent each overlaps with that of melanopsin. The spectral sensitivity function is the degree to which a given photopigment responds (y-axis) to a photon of light of a particular wavelength (x-axis).

FIG. 2 depicts one non-limiting embodiment of a system of the present disclosure, where a sinusoidal modulation of light is presented to a pharmacologically dilated eye from a digital spectral integrator (1), and the consensual pupil response is measured in the fellow eye with an infrared camera (2). The digital spectral control and infrared camera can be controlled by a control and analysis software means (3).

FIG. 3 depicts modulations of the spectra of light designed to target specific photoreceptors via “silent substitution.” The device produces light with a mean background spectrum (depicted in gray). The spectrum of the light then modulates around this background in one embodiment, engaging in a smooth, sinusoidal modulation between a positive and a negative spectrum, traversing the intermediate spectral forms over time. The particular spectra chosen have the theoretical property of stimulating a particular photoreceptor and not the others. The spectrum in black is termed the “positive” spectrum. It has the theoretical property of increasing photon capture in the targeted photoreceptor relative to the background, but not altering photo capture in the non-targeted (“silenced”) photoreceptors. In gray is the “negative” spectrum. It has the theoretical property of decreasing photon capture in the targeted photoreceptor relative to the background, but not altering photo capture in the non-targeted (“silenced”) photoreceptors. Modulating the light from the positive, to background, to negative spectra and back again nominally varies the response of the targeted photoreceptor over time, while not altering the response of the non-targeted photoreceptors.

FIG. 4 depicts the predicted photoreceptor responses to a nominally S-directed spectral modulation. At the top is a modulation designed to target S-cones using the standard templates of photoreceptor sensitivities. It has the nominal property of producing greater and lesser stimulation of S-cones over time, resulting in 45% contrast on these targeted photopigments. The other photoreceptors (melanopsin, M, and L cones, have 0% predicted contrast).

FIG. 5 depicts how normal biological variation can alter the spectral sensitivity of a silenced photopigment. The spectral sensitivity of melanopsin is shown, and is seen to vary depending upon the wavelength of peak sensitivity (λmax) and the observer age. Observer age produces variation in the spectral filtering of light reaching the eye. A modulation, for example, designed to target S-cones and silence melanopsin will produce inadvertent stimulation of melanopsin if the assumed biological properties of the observer are not perfectly realized. Spectral sensitivity estimates of melanopsin were obtained using the Stockman-Sharpe nomogram, a field size of 10°, adjusting for pre-receptoral filtering according to the CIE standard for cone fundamentals using the observer age parameter (20-80 years), and assuming a peak optical density of 0.3. Wavelength of peak sensitivity λmax was varied between 470 and 490 nm.

FIG. 6 depicts the standard S-directed modulation and the “robust” S-directed modulation that is produced by the methods and systems of the present disclosure. The robust modulation has the property of reduced sensitivity to biological variation as compared to the standard modulation. This is achieved by silencing not just the non-targeted photoreceptors, but by silencing hypothetical versions of the non-targeted photoreceptors that have different peak spectral sensitivity or have been subjected to differential pre-retinal filtering. Both modulations produce a nominal 45% contrast on S-cones.

FIG. 7 depicts the improvement in unwanted contrast on non-targeted photoreceptors achieved by the methods and systems of the present disclosure. Each plot shows the calculated contrast upon melanopsin created by a nominally melanopsin silencing, S-targeted modulation. A 32 year-old observer is depicted. The x-axis considers a deviation in the actual pre-retinal filtering of the subject (expressed in years) from the assumed age. The y-axis considers a deviation in the actual λmax of melanopsin from the assumed 480 nm. The degree of inadvertent contrast upon melanopsin under these forms of biological variation is expressed by the color scale. The overall degree of unwanted melanopsin contrast is reduced by the robust isolation. The ellipses trace the melanopsin contrast associated with ±2 and ±3 standard deviations of the expected population variation in the CIE age parameter (standard deviation estimated as 7 years) and variation in the λmax of melanopsin. A standard deviation of 1.5 nm was assumed for melanopsin (which is the maximum of the variability estimated for the human L, M, and S cone classes).

FIG. 8 depicts the improvement in unwanted contrast on non-targeted photoreceptors achieved by the methods and systems of the present disclosure as a summarized table. The maximum and minimum unwanted contrast on melanopsin (for an S-directed modulation) is given for 2 standard deviations (95% confidence interval) and 3 standard deviations (99% confidence interval) of biological variation in pre-retinal filtering and λmax of melanopsin. Unwanted contrast is reduced by over a factor of 4 by the robust modulation as compared to standard techniques.

FIG. 9 depicts an application of the methods and systems of the present disclosure. A 0.1 Hz spectral modulation is presented to an observer. The consensual pupil response is recorded over many trials and averaged.

FIG. 10. A least-squares spectral fit is performed to measure the amplitude of pupil response at the stimulation frequency.

FIG. 11A-B. (a) A least-squares spectral fit is performed to measure the amplitude of pupil response at the stimulation frequency. (b) This can be repeated for a variety of photoreceptor-directed modulations and at different temporal frequencies of modulation. The average response across cycles of the modulation is depicted for a single human observer. This illustrates that measurable responses from human observers can be obtained for the different photoreceptors and at different temporal frequencies.

FIG. 12. A sinusoid fits the data from human observers well, and allows the response to be characterized by two numbers: the amplitude of the response and the phase (or timing).

FIG. 13. The data can be summarized in a polar-plot, which expresses amplitude as distance from the center and phase as position around the circle. The data from each subject can be normalized by expressing the response to each photoreceptor modulation as proportional to the complex sum of responses across photoreceptor classes obtained from that observer.

FIG. 14 presents data from 16 human observers studied with a 0.5 and 0.05 Hz modulation of S, L+M, and melanopsin targeted modulations. The L+M and melanopsin driven responses are shown. A measurable melanopsin response is seen. (Upper) L+M and melanopsin responses are in phase at 0.5 Hz. Each plot point corresponds to one human observer. (Lower) L+M and melanopsin responses are in quadrature phase at 0.05 Hz. This illustrates 1) the robust isolation of the technique and 2) the ability of relative measurement (scaling by the total response) to remove incidental individual differences, creating the power to measure clinically relevant departures from this control group.

FIG. 15 presents data from 16 human observers. The L+M and melanopsin responses from the previous plot have been summed into a “brightness” response and are plotted in yellow. The responses from an S-cone targeted modulation are shown in dark gray. Again, the tight-clustering of responses across observers illustrates the power of the present disclosure to make reliable measures from individual human observers, and to separately probe the responses of the different photoreceptors.

FIG. 16 presents data from 16 human observers. Each plot point indicates the L+M and melanopsin responses for a given observer. The strong correlation between these measures illustrates the benefit to be gained in measurement for individuals by scaling measured responses by the total response.

FIG. 17A-B depicts measurements of pupil response made for the different photopigment isolations at several different temporal frequencies of modulation, resulting in modulation transfer functions for each photoreceptor type. This is done for two human subjects. The different shapes of response seen across temporal frequencies illustrates the separate isolation of photoreceptor types by the approach.

FIG. 18. In BOLD fMRI studies, photoreceptor targeted spectral modulations were flickered at a range of modulation frequencies. A given modulation frequency was presented within a 16 second epoch, and the order of modulation frequencies was counterbalanced. The subject observed the spectral modulations through an artificial pupil while BOLD fMRI scanning measured neural responses from cortical visual area V1.

FIG. 19. The neural responses for three observers were obtained from within area V1 across the range of flicker frequencies and for different targeted photoreceptor classes (or their combination). The average and standard error of the mean of evoked neural responses are given, and distinct temporal transfer functions were observed for the different photoreceptor classes and chromatic channels.

FIG. 20A-C depicts selective targeting and silencing of penumbral cones. (a) Light passes through blood vessels before striking some cones on the retina. This light is subjected to spectral filtering by hemoglobin. This can result in unwanted contrast on non-targeted photoreceptors at some points on the retina. (b) Illustration of the power of the robust-targeting technique. A robust modulation was created that silenced all photoreceptors in the retina excluding only those L and M cones located in the penumbral shadow of blood vessels. When this modulation was viewed by a human observer, this allowed her to see her retinal blood vessels and produce the sketch shown. The similarity of this sketch to the vascular anatomy of the retina is readily apparent. In addition to selectively targeting penumbral cones, they can be silenced. (c) The plot shows the measured pupil response of a human observer to melanopsin stimulation with and without penumbral cone silencing. The similar result indicates that substantial contrast on this targeted photoreceptor can be maintained while rendering the stimulus robust to biological variation across location in the retina.

FIG. 21A-C depicts an application of the methods and systems of the present disclosure to measure the responses of the visual system at high temporal frequencies using pupillometry. (a) Amplitude modulation of spectral variation created to target a photoreceptor. A high-frequency carrier modulation is itself modulated by a low-frequency carrier wave. (b) Viewing this stimulus produces a measurable pupil response in a human observer, illustrating the capability to measure a response through the pupil of high-temporal frequency retinal physiology. (c) This measurement is repeated here for one human observer over a range of carrier-frequencies and for two photoreceptor-targeted modulations. The resulting modulation transfer functions illustrate the capability of the approach to measure robust, photoreceptor specific responses to high temporal frequency modulations.

FIG. 22A-B. S input to the pupillary light response is opponent to L+M and melanopsin. (a, top) Stimulus modulation over time between positive and negative spectra. (a, middle) Pupil traces for two 120-s trials (sub01, 0.05 Hz, L+M). (a, bottom) Average data (12 trials; same subject/condition; first 20 s discarded) fit with a sinusoid at the stimulus fundamental. (b) One cycle of the pupillary light response [sub01 and group average over 16 subjects (black lines); melanopsin responses ×2 scale, S responses ×3]. Dashed lines show fit with fundamental. Where visible, magenta lines are the fits with fundamental and second harmonic.

FIG. 23A-B. Group pupillary light response data are well fit by the two component linear filter model. (a) The mean response across all subjects (01-16) is shown at 0.05 and 0.5 Hz, for L+M-, melanopsin-, and S-cone-directed modulations. Fit values were derived from those found for subject 01, with only amplitude parameters adjusted. This is because the average data are available at only two temporal frequencies and do not sufficiently constrain all parameters of the model. To obtain the average data plotted, amplitudes and phases were averaged separately (i.e., average amplitude obtained without consideration of phase, average phase obtained without consideration of amplitude). The model was fit to the data as plotted. (b) Polar-plot representations of the group data with model fit points, following conventions as in FIG. 17. The data were normalized separately for each temporal frequency. Error bars (+/− SEM across subjects) are smaller than the plot points for the data.

FIG. 24 shows the across-cycle average pupil response from a single subject viewing slow (48 seconds per cycle) square-wave, isochromatic (non-color changing) spectral modulations. These modulations include a light flux modulation in which power in the entire visible spectrum of light is raised and lowered, a cone (L+M+S) directed modulation, and a melanopsin directed modulation. A distinct temporal profile is observed for the cone and melanopsin directed modulations, confirming their physiologic separation by this method. Notably, the response to the light flux modulation is well modeled (dash line) by the sum of the cone and melanopsin responses.

FIG. 25 shows the mean brightness rating obtained across trials and subjects as a function of cone and melanopsin contrast. Six subjects provided ratings on a 1-100 scale of perceptual brightness of a spatially uniform field. After adapting to a background spectrum, on each of many trials the observer was presented with a test spectrum for 4 seconds and was asked to rate the brightness of the spectrum. Each spectrum was generated by a crossing of positive and negative changes in cone (L+M+S) and melanopsin contrast, ranging from +40 to −40% contrast. Onset and offset of the spectrum graded by a 0.5 second half-cosine ramp, further masked with low-contrast, chromatic “cone noise.” The plot shows the brightness ratings as a function of melanopic contrast, with separate lines for measurements at each cone contrast level. The results demonstrate that both cone and melanopic contrast contribute additively to a report of brightness of the field.

FIG. 26 shows the amplitude of electromyographic (EMG) signal from the orbicularis oculi muscle of observers while they performed the brightness rating task presented in FIG. 25. The electrical activity of the orbicularis oculi indexes the degree of eyelid “squint,” which is an involuntary response to visual discomfort. The plot shows an increasing amount of eyelid squint with increasing levels of melanopic contrast.

### DETAILED DESCRIPTION OF THE DISCLOSURE

The present disclosure relates to systems and methods that can be used to stimulate and record responses elicited from naturally-occurring or artificially-introduced photosensitive molecules.

System for the Rapid, Robust and Non-Invasive Stimulation of Photopigments and Photosensitive Molecules

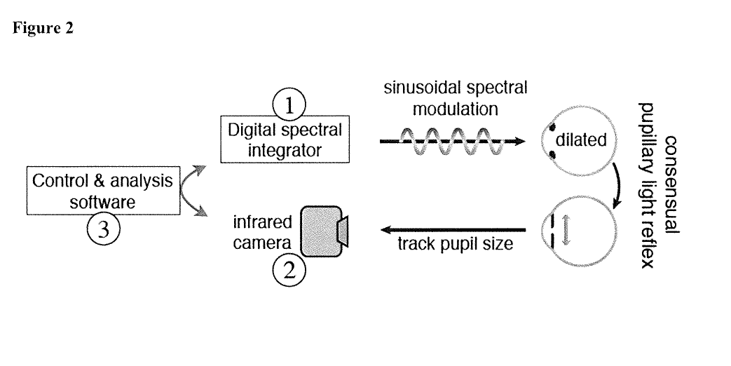

For the purpose of illustration and not limitation, FIG. 2 is a schematic representation of an exemplary system according to the disclosed subject matter. In certain embodiments, the systems of the present disclosure can include: (1) a digital spectral integrator, e.g., a light source, and (2) a response detection means.

In certain non-limiting embodiments, the digital spectral integrator, e.g., light source, can be a device that produces light of a specified spectrum and/or temporal profile. In certain embodiments, the light of a specified spectrum and/or temporal profile is a field of spatially uniform light. In certain embodiments, the light source can be a tailored spectrum of light using a Digital Light Processing (DLP) chip that uses digital micro-mirrors to vary the intensity of light collected from different wavelength bands. In certain embodiments, the digital manipulation of light spectra via a DLP chip can be performed using a device manufactured by OneLight Corp. (http://www.onelightcorp.com). Alternative light sources, such as single or multi-channel light emitting diode (LED) light sources, can also be used in connection with the systems described herein.

In certain embodiments, the light output from the light source is presented to a pharmacologically dilated eye of a subject. Alternatively or additionally, the light output can be presented to the subject through an aperture, e.g., an aperture with a diameter of about 2 mm to about 5 mm, e.g., to equalize retinal irradiance across subjects. For example, and not by way of limitation, the aperture can have a diameter of about 2 mm to about 3 mm, about 3 mm to about 4 mm, about 4 mm to about 5 mm, from about 2 mm to about 4 mm or from about 3 mm to about 5 mm. In certain embodiments, an eyepiece can be employed through which the subject views the stimulus. The eye piece can be composed of conventional optical components.

In certain embodiments, the digital spectral integrator can produce a spectral modulation that includes a temporal modulation at a carrier frequency, e.g., a sinusoidal carrier frequency. For example, and not by way of limitation, the carrier frequency can be from about 0.01 Hz to about 128 Hz. In certain embodiments, the carrier frequency can be from about 0.1 Hz to about 128 Hz, from about 1 Hz to about 128 Hz, from about 10 Hz to about 128 Hz, from about 50 Hz to about 128 Hz, from about 100 Hz to about 128 Hz, from about 0.01 Hz to about 100 Hz, from about 0.01 Hz to about 50 Hz, from about 0.01 Hz to about 10 Hz, from about 0.01 Hz to about 1 Hz or from about 0.01 Hz to about 0.1 Hz.

In certain embodiments, the digital spectral integrator can produce an amplitude-modulated light that includes a lower envelope frequency and/or a higher carrier frequency. In certain embodiments, the higher carrier frequency can be from about 4 to about 128 Hz, e.g., from about 4 to about 100 Hz, from about 4 to about 50 Hz, from about 4 to about 10 Hz, from about 10 to about 128 Hz, from about 50 to about 128 Hz or from about 100 to about 128 Hz. In certain embodiments, the lower envelope frequency can be from about 0.01 to about 1 Hz, e.g., from about 0.01 to about 0.5 Hz, from about 0.01 to about 0.1 Hz, from about 0.01 to about 0.05 Hz, from about 0.1 to about 1 Hz or from about 0.5 to about 1 Hz.

In certain embodiments, the detection means can be an infrared camera. For example, but not by way of limitation, the detection means can be an infrared camera capable of measuring the consensual pupillary response of the fellow eye to the light modulation. Such infrared camera technology is in general use and devices are sold by multiple manufacturers, e.g., Cambridge Research Systems (http://www.crsltd.com), and are supplied with software that automatically measures the pupil diameter from the infrared images. Alternatively or additionally, the detection means can include a functional magnetic resonance imaging (MRI) scanner or electroencephalogram for the measurement of a visual evoked potential (VEP), e.g., for the detection of the neural activity of the brain of the subject being tested.

In certain embodiments, the detection means can be essentially any apparatus capable of measuring a response elicited by stimulation of a photosensitive molecule. For example, by not by way of limitation, an electroretinogram (ERG) can be used to measure a response, e.g., by detecting the electrical response of the retina. In addition, in contexts where the response elicited by stimulation of a photosensitive molecule is apparent to a subject being stimulated, e.g., in the context of photophobic pain, the subject can act as the detection means by relaying their perception of the response. In addition, the reflexive response of a subject to photophobic pain, e.g., eyelid squint, may serve as a detection means.

In certain embodiments, the system of the present disclosure can further include an integration means, e.g., a control and software means (see the non-limiting exemplary embodiment depicted in FIG. 2). For example, and not by way of limitation, a system of the present disclosure can include (1) a digital spectral integrator, e.g., a light source, (2) a response detection means, e.g., an infrared camera, and (3) an integration means, e.g., a control and software means. In certain embodiments, the integration means (3) is coupled to the digital spectral integrator (1) and/or the response detection means (2).

In certain embodiments, the integration means can include, but is not limited to, a computer software program capable of instructing the light source to emit particular variations of light over time. For example, and not by way of limitation, the computer software program can be capable of instructing the light source to emit modulations of light that stimulates a particular photosensitive molecule, e.g., melanopsin. The computer software can also be capable of interpreting the response to the stimulus, e.g., pupil response, detected by the detection means. For example, and not by way of limitation, the integration means can be used to provide on-line control of the light source to create stimuli that are conditional to and adaptive to the response.

In certain embodiments, the system can employ computer software, e.g., as the integration means, to instruct the light source to produce a particular kind of variation of light over time and to record the detection means measure of a response, e.g., pupil dilation and/or movement, to this light. In the context of detecting responses to melanopsin stimulation, the light modulation is identified as “robust melanopsin isolating.” In certain embodiments, the response of a subject can be measured for a set of carrier frequencies and photoreceptor classes.

The systems of the present disclosure are also capable of measuring responses to other modulations of light, for example, but not by way of limitation, for comparison purposes. In this way, the system and approach of the present disclosure can be used to not only create a robust stimulus but also to interpret the corresponding response, including responses relative to other types of light stimulation, e.g., as an individual difference measure.

In certain embodiments, including, but not limited to those involving photosensitive retinal cell stimulation, the systems of the present disclosure can involve the technique of silent substitution. The technique of silent substitution allows the selective stimulation of a class of photopigments or photosensitive molecules. In ophthalmologic practice, this isolation requires precise specification of many biological properties of the eye. This includes the peak spectral sensitivity of the photopigments (which is subject to genetic variation) and variation in observer age (which varies the spectral properties of the lens of the eye). In certain embodiments, the methods and systems of the present disclosure provide for the ability to create silent substitution stimuli that are simultaneously powerful and robust to these variations.

System Applications

The present disclosure further provides methods of using the disclosed system. Certain embodiments of the present disclosure relate to measurement of biological responses to isolated stimulation of particular photopigments. In another aspect, methods of the present disclosure relate to producing a biological effect in a subject using the disclosed system.

In certain embodiments, the present disclosure relates to a method of detecting a response and/or producing a biological effect associated with selectively stimulating a photosensitive molecule, e.g., a photopigment, including (a) contacting the photosensitive molecule with a light of a specified spectral and temporal profile and (b) detecting said response associated with selective stimulation of the photosensitive molecule. For example, but not by way of limitation, such photopigments can be found in the photosensitive cells of the eye of a subject, such as, but not limited to, the L, M and S-cones and melanopsin. In certain embodiments, the light of a specified spectral and temporal profile can be provided as a spatially uniform field of light. Additional certain non-limiting embodiments can include the use other spatial profiles. For example, but not by way of limitation, a sinusoidal spatial variation of the stimulation can be used and the spatial frequency of this sinusoidal variation can be varied to measure the spatial integration properties of the response to the targeted photosensitive molecules. In certain embodiments, a square wave, e.g., non-sinusoidal, variation of the stimulation can be used.

A “subject” or “patient,” as used interchangeably herein, refers to a human or a non-human subject. Non-limiting examples of non-human subjects include non-human primates, dogs, cats, mice, rats, guinea pigs, rabbits, pigs, fowl, horses, cows, goats and sheep.

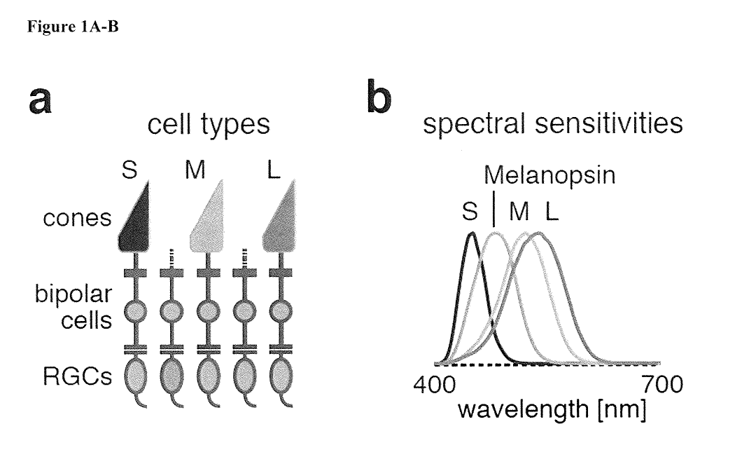

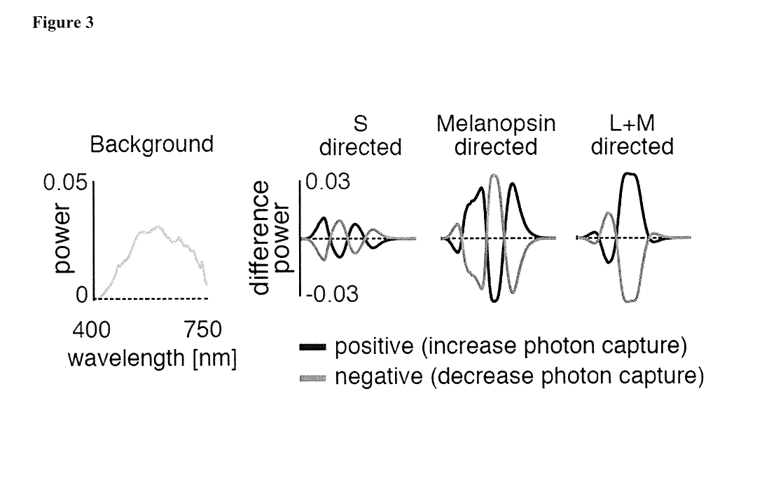

In certain embodiments, the photosensitive cell type of the eye is the S-cone. The spectral sensitivity of S-cones overlaps with that of melanopsin (see FIG. 1A). A modulation using the presently disclosed system can be created that nominally stimulates S-cones and silences melanopsin. Misspecification, however, of the peak spectral sensitivity of melanopsin, or misspecification of the lens density of the subject, can result in the modulation that produces unwanted melanopsin stimulation, and thus confuse the biological measure. Using the systems and methods described herein, a robust stimulus modulation can be created (see FIG. 3) that is insensitive to these incidental biological variations and produces more complete S-cone isolation. For example, and not by way of limitation, a method of the present disclosure can include robustly silencing responses from photosensitive molecules that are not targeted by the stimulus despite the biological variation in the spectral sensitivities of the photosensitive molecules.

In certain embodiments, enhanced control over the spectral and temporal properties of light provided by a digital spectral integrator to allow a modulation of light that stimulates melanopsin and, advantageously, will minimally stimulate other light-sensitive cells of the eye, can be created. In certain embodiments, the present device can be used to silence cones not shadowed by the blood vessels to produce isolated stimulation of melanopsin. The disclosed methods are advantageous in that the resulting measurement is robust to individual differences in the realized spectral sensitivity of melanopsin and other photopigments related to one or more of the following: age of the subject; differences in eye pigmentation; differences in anterior segment pathology (e.g., cataracts); differences in absolute light level reaching the retina; loss of fixation stability; and polymorphisms in genes that code for the photopigments. For example, and not by way of limitation, the disclosed methods allow creation of stimuli that are insensitive to the location of the stimuli across the retina, and does not require a subject to maintain fixation while the stimulation is isolated, which can be difficult for subjects suffering from a loss of central vision or nystagmus.

In certain embodiments, the pupil response to melanopsin stimulation can be compared to the response elicited by stimulation of the L and M cones. This relative response has been found to vary in systematic ways (amplitude and timing) across individuals. In certain embodiments, the pupil response of a patient can be measured for a set of carrier frequencies and photoreceptors. In certain embodiments, the systems of the present disclosure can express a scaled melanopsin sensitivity score based upon a comparison of multiple measures that stimulate different photoreceptor pathways. This innovation can allow for the measurement of individual differences in melanopsin response independently of overall differences in pupil response (e.g., resulting from factors such as, but not limited to, patient variation in age and iris pigmentation). The details of this response interpretation can then be informed by accumulating additional samples of control and clinical subject data.

The present system has uses beyond simply detecting changes in melanopsin sensitivity. For example, but not by way of limitation, melanopsin sensitivity has been implicated in several physiologic functions and corresponding clinical conditions. Accordingly, the systems of the present disclosure can provide an objective measure of the disruption of the melanopsin pathway in clinical psychiatric, neurologic and/or behavioral diseases that either lack such an objective measure or where such a measure is less reliable than what use of the present system can provide. In certain embodiments, the psychiatric disorder can include seasonal affective disorder and other mood disorders. In certain embodiments, the neurological disorder can include migraines, photophobia, traumatic brain injury and neurodegenerative disorders involving the brainstem, such as, but not limited to, Progressive Supra-nuclear Palsy. An additional non-limiting example of an application of the system includes obtaining a biomarker of melanopsin sensitivity in photophobia to guide drug development and administration of a melanopsin-specific chemical agonists and antagonists. Disease states relevant to such a biomarker include, but are not limited to, migraine, post-concussive photophobia, uveitis, mood disorders, seasonal affective disorder and sleep disorders.

In certain embodiments, a system of the present disclosure can be used to detect impairments in the temporal sensitivity of photosensitive cells, which are early feature of ophthalmologic diseases and/or disorders. Non-limiting examples of ophthalmologic diseases that can be detected using the present disclosure includes inherited retinal degenerative diseases, e.g., retinitis pigmentosa and glaucoma, and macular degeneration. For example, and not by way of limitation, a system of the present disclosure can be used to assess the functional state of photosensitive cells, such as, but not limited to, retinal ganglion cells (ipRGCs), rods and cones, prior to, during or subsequent to, the provision of prosthetic, optogenetic or gene-therapeutic treatments, e.g., treatments for degenerative retinal disease, including inherited conditions such as Leber's Congenital Amaurosis and acquired disorders such as macular degeneration or diabetic retinopathy.

While the present application describes the disclosed system largely in the context of stimulating photosensitive retinal cells and measuring ocular responses to such stimulation, systems capable of implementing the techniques described herein can take many forms and be employed to detect a wide variety of responses. For example, and not by way of limitation, the systems of the present disclosure can be employed to detect measurements of perception, such as flicker fusion frequency, including those related to visual discomfort produced by high-frequency flickering lights. In certain embodiments, the measurement of perception can include EMG of squinting. In certain embodiments, the systems of the present disclosure can be used to stimulate and measure the pupil response from a single eye using adaptive stimulus control, in contrast to measuring the consensual response in the non-stimulated eye. In certain embodiments, the systems of the present disclosure can be used to stimulate and measure the cortical and thalamic response.

The systems of the present disclosure can also be employed to provide an assessment of high temporal frequency to detect disorders of retinal function that manifest as a loss of high temporal frequency function. For example, and not by way of limitation, the system can produce an amplitude-modulated flicker of light that has a lower envelope frequency and a higher carrier frequency to allow the measurement of the modulation transfer function at temporal frequencies higher than the pupil can respond to directly, e.g., by pupil movement. In certain embodiments, the higher carrier frequency can be from about 4 to about 128 Hz, e.g., from about 4 to about 100 Hz, from about 4 to about 50 Hz, from about 4 to about 10 Hz, from about 10 to about 128 Hz, from about 50 to about 128 Hz or from about 100 to about 128 Hz. For example, and not by way of limitation, the amplitude-modulated stimuli (e.g., flicker of light) can be presented at four carrier frequencies such as 5 Hz, 10 Hz, 20 Hz and 40 Hz. In certain embodiments, the envelope frequency can be from about 0.01 to about 1 Hz, such as, but not limited to, about 0.5 Hz. The spectral modulations of the flicker of light can be tailored to selectively stimulate particular photopigments, e.g., melanopsin. In certain embodiments, amplitude of the pupil response at different carrier frequencies can be measured in response to the flicker of light to assess the temporal response properties of the different photopigments. In certain embodiments, the spectral modulations of the carrier frequency and envelope frequency can differ. For example, but not by way of limitation, the carrier frequency can stimulate the L and M cones selectively, while the envelope frequency can stimulate melanopsin selectively. The systems of the present disclosure can be capable of encoding multiple stimulus modulations targeted at single or multiple photopigments at different simultaneous temporal frequencies.

In certain embodiments, the system of the present disclosure can be used to specifically target single or multiple photopigment classes that are naturally-occurring or have been artificially introduced. In particular, the system of the present disclosure can be used to selectively stimulate optogenetic photosensitive proteins that are introduced into the neural tissue of the subject such as, but not limited to, the tissue of the central nervous system and/or the peripheral nervous system. In certain embodiments, the neural tissue is the retina. Non-limiting examples of optogenetic photosensitive proteins that can be introduced into the neural tissue of a subject include halorhodopsin, channelrhodopsin and archaerhodopsin. Additional non-limiting examples of photosensitive proteins are described in Yizhar et al., Neuron 71:9-34 (2011) and Zhang et al., Cell 147:1446-1457 (2011), which are incorporated by reference herein in their entireties.

Stimulation of photopigments, e.g., artificially introduced optogenetic photosensitive proteins, can be used to produce a biological effect and/or for the purpose of controlling neural activity and behavior. For example, and not by way of limitation, stimulation of photopigments can be used to restore visual function and/or treat psychiatric, neurologic and/or behavioral diseases. In certain embodiments, stimulation of photopigments can be used to treat retinal disease, macular degeneration, photophobia, Leber's Congenital Amaurosis, retinitis pigmentosa, mood disorders, seasonal affective disorder and/or sleep disorders.

The systems of the present disclosure can also be employed as a stimulus and response interpretation strategy in electroretinograms (ERGs) as well as a stimulus and response interpretation strategy in neuroimaging (e.g., fMRI) measurements. In certain embodiments, the systems of the present disclosure can also be employed as a stimulus and response interpretation strategy in visual evoked potentials (VEP).

While the above-described applications can be accomplished through the use of the system as described herein, devices for employing the systems and/or methods of the present disclosure can be miniaturized and packaged to join the suite of tools commonly available in a conventional ophthalmologic examination room.

The following examples are merely illustrative of the presently disclosed subject matter and should not be considered as limitations in any way.

### EXAMPLES

### Example 1

### Detecting Differences in Responses Elicited by Photoreceptor Directed Light Stimulation

The purpose of this Example is to examine the behavioral, physiologic and neural response of human subjects to photoreceptor directed light stimulation. The eye is composed of several different light sensitive cells (rods, cones, intrinsically photosensitive retinal ganglion cells—ipRGCs). These different cell types are used both for visual perception, as well as non-visual functions of the eye (control of pupil size and circadian rhythm). These functions are altered in some disease states. Using a device that presents modulations of spectra of light under digital control, subjects viewed the stimulus while making responses to perceptual questions, having their pupil size monitored with an infrared camera or undergoing functional MRI brain imaging. These studies can be conducted in a not control population, and patient populations with disorders hypothesized to interact with photoreceptor subtypes, e.g., patients with migraine headaches and retinal disease.

Background:

The retina is composed of a sensory layer (containing rods and cones, also called the outer retina) and a neural layer (containing bipolar and ganglion cells, also called the inner retina). Recently, and surprisingly, a subset (1-3%) of retinal ganglion cells have been found (Provencio et al. 2000) to be themselves light-sensitive (ipRGCs). The photopigment melanopsin provides this sensitivity, with a peak spectral sensitivity of 480 nm (in the blue range, similar to rods) and a temporal integration over many seconds. The ipRGCs are thought to mediate non-image-forming visual functions via sub-cortical projections, including entrainment of circadian rhythms (Morin et al. 2003, Lucas et al. 1999); pupillary control (Gamlin et al. 2007); and photophobia (painful light sensitivity) (Noseda et al. 2010). The last of these appears driven by the common projections of the trigeminal nerve and the ipRGCs to the posterior thalamus. These exciting findings from animal studies, however, have seen limited extension to human neuroimaging due in part to two barriers (although see Vandewalle et al. 2007).

First, outside of genetically modified mice born without rod and cone function, it is difficult to selectively stimulate melanopsin, given the overlap of melanopsin spectral sensitivity with that of the cone photopigments. Second, unlike single-unit studies in animals, fMRI in people requires knowledge of the temporal integration properties of ipRGCs to measure neural responses. These challenges must be addressed if melanopsin-driven responses are to be measured in the human central nervous system, and then tested for altered responses in diseases such as seasonal affective disorder (Roecklein et al. 2009), migraine (Noseda et al. 2010), and inherited retinopathies (Aguirre et al. 2007). Allelic variations in the gene that encodes melanopsin (OPN4) have been associated with variations in the pupillary response to light (Higuchi et al., PLOS-ONE, 2013). This Example uses a digital, spectral-controlled light source to: (1) produce photoreceptor directed light modulations via silent substitution (see Estévez and Spekreijse 1974; and Vienot and Brettel 2014); (2) measure responses from normally sighted, human subjects to this light modulation using pupillometry, behavioral assays of perceptual sensitivity and neural responses using functional MRI; and (3) compare these measures made in control populations to patients with a variety of disorders hypothesized to interact with variations in outer and inner retinal photoreception.

Design:

Photoreceptor isolation by silent substitution and behavioral and pupil measures. The photopigments (L, M, and S cones and melanopsin) have different sensitivity profiles to light of different wavelengths. It is possible to pick two spectra of light such that the absorption of photons from the two stimuli is equal for the cone photopigments, but different for melanopsin (Brainard et al. 2010; Tsujimura et al. 2010; Estevez and Spekreijse 1974; and Vienot and Brettel 2014). Rods can be silenced by presenting the spectra in the photopic range. Modulating between stimuli with the background and isolating spectra will stimulate, for example, ipRGCs to a greater and lesser degree, while holding cone and rod stimulation constant.

The stimuli were generated using a OneLight Spectra Digital Light Engine (http://www.onelightcorp.com/products/onelight-spectra/), which produces a wide-field)(25°), spatially uniform field of a specified spectral profile in the visible range under computer control. In particular, a white light source (a xenon light bulb) was passed through a refraction system to tune the spectrum of light (e.g., more red light, or more blue light). The subject viewed the field of light through an eye piece. The mean luminance of the stimulus was on the order of 2500 cd/m², which is quite comfortable to view (roughly equivalent to the luminance of the full moon). Lower or higher mean luminances can also be used. All light levels were below the relevant ANSI (http://www.ansi.org) and ISO (http://www.iso.org) safety standards. Subjects viewed the stimulus through an eye piece using one eye. The eye piece passed the light through a diffusing lens and back-projected the light on a translucent plastic disc which the subject views. Control of pupil size in the stimulated eye was achieved either by pharmacologic dilation and/or by having the subject view the stimulus through a small (e.g., about 2 to about 5 mm diameter) aperture (an artificial pupil). The phase and amplitude of the consensual pupillary response to a photoreceptor-isolating, spectral modulation of different temporal frequencies was measured.

From each of several subjects, automated infrared pupillometry (at 50 Hz) were obtained from the one eye (Video EyeTracker, Cambridge Research Systems Ltd) while the other eye views spectral modulations through an eye piece. The amplitude of pupil size modulation was measured at different temporal frequencies of stimulation. The resulting transfer function characterizes the temporal integration properties of the given photoreceptor pathway, a fundamental physiologic measure of the system. In other studies, the contrast, temporal frequency, or direction of photoreceptor modulation of the stimulus can be varied in a staircase fashion to determine the threshold at which a specified level of accuracy can be achieved in discriminating the presence of absence of the modulation under study.

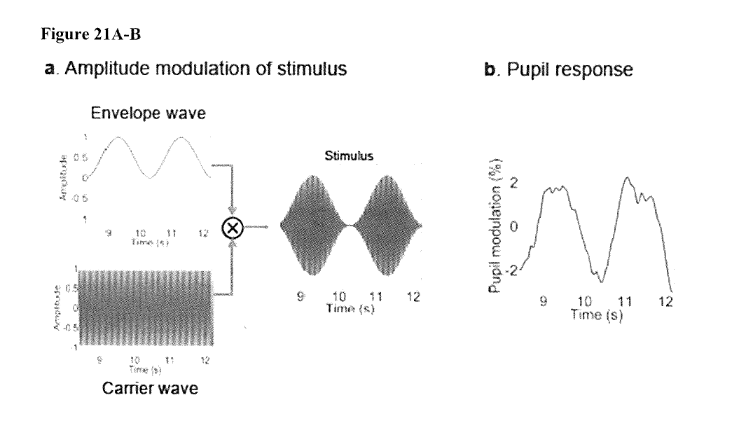

BOLD fMRI. Each of several subjects viewed spectral modulations while undergoing Blood Oxygen Level Dependent (BOLD) fMRI at 3T using a 32 channel head coil. Stimulation can be provided at a lower temporal frequency (<0.25 Hz) with the goal of measuring the direct modulation of the amplitude of neural firing evoked by the stimulus. Alternatively, stimulation can be provided in brief (e.g., 16 second) epochs during which the stimulus will be flickered at a higher temporal frequency (>0.25 Hz) (FIG. 20). Higher frequency flicker was used to measure the non-linear neural response to flicker carried by ON and OFF sensitive neurons. Custom Matlab code synchronized the timing of photoreceptor directed stimulation with image acquisition by the scanner. Subjects viewed the stimulus through an MRI compatible eye piece. Periods of photoreceptor isolating stimulation was alternated with the background spectrum as a control. Each subject was scanned during a 1-2 hour session featuring multiple presentations of the stimuli in counter-balanced order.

Genotyping. Genotyping studies will involve collection of DNA samples using the noninvasive collection method of mouthwash rinse into a vial or cheek swab for genotyping purposes. DNA extraction and sequencing will be performed in the Molecular Biology Core Facility of the Perelman School of Medicine at the University of Pennsylvania. The OPN4 polymorphism of rs1079610 (I394T) will be genotyped using the TaqMan SNP Genotyping Assay (Applied Biosystems, Foster City, Calif.) according to the manufacturers procedure. Genotyping Assay ID is C_{—}1736425-1 for rs1079610 (I394T).

Results:

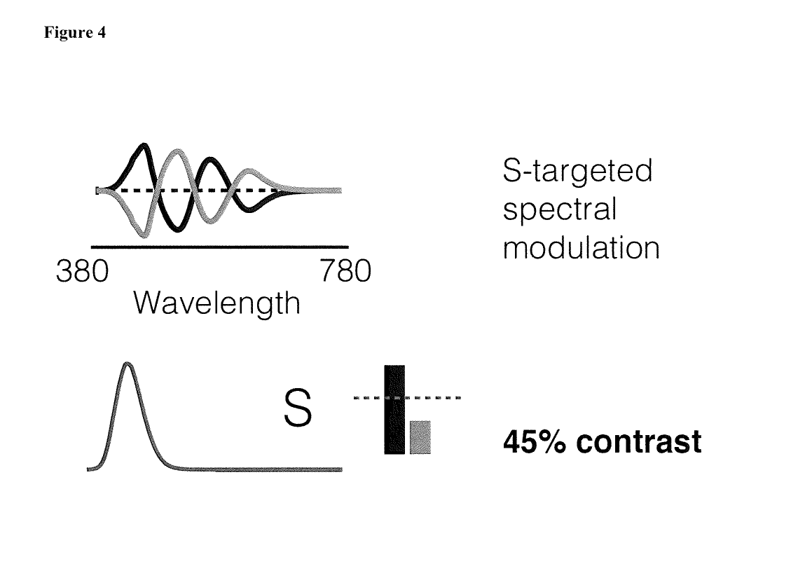

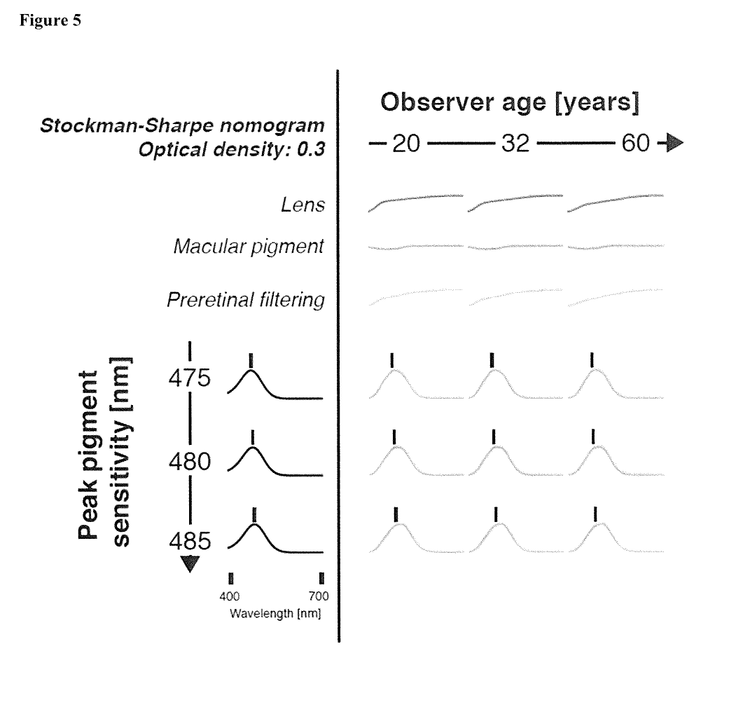

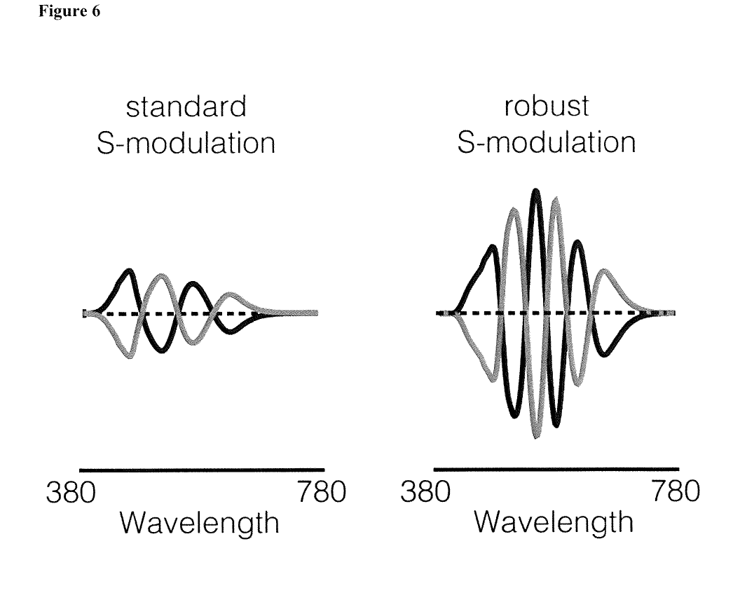

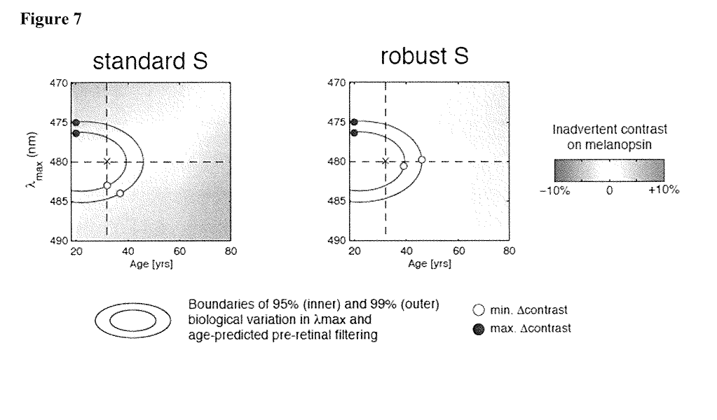

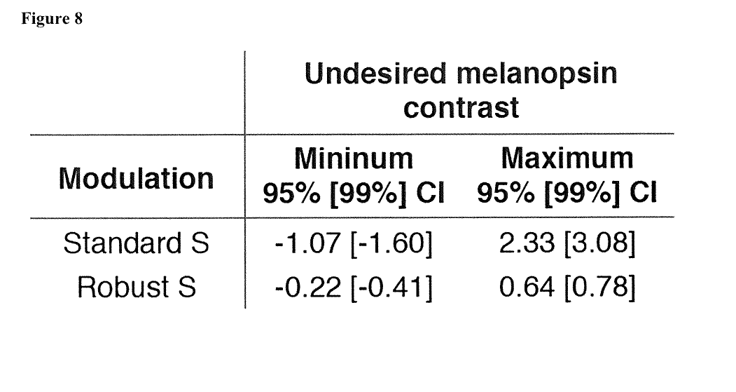

Robust spectral modulations reduce sensitivity to unwanted biological variation. Spectral modulations around a background spectrum can be used to targeted classes of photoreceptors using silent substitution (FIGS. 3 and 4). This technique relies upon accurate specification of the spectral sensitivity function of the targeted and silenced photoreceptors. Biological variation in, for example, the genetically determined peak spectral sensitivity of photopigments or the density of the lens of the eye related to subject age can alter the realized spectral sensitivity of a targeted or silenced photopigment (FIG. 5). The present disclosure describes the robust targeting of photopigments to reduce this sensitivity to unwanted biological variation. Robust modulations use a greater number of spectral primaries to silence a photoreceptor with the canonical spectral sensitivity profile, but also hypothetical variations in spectral sensitivity produced by biological variation (FIG. 6). Effectively, robust targeting amounts to silencing not just a photoreceptor, but simultaneously silencing a family of hypothetical photoreceptors with variations in spectral sensitivity. This robust targeting results in decreased inadvertent contrast upon non-targeted photoreceptors in the presence of biological variation in, for example, peak spectral sensitivity or subject age (FIG. 7). In the example of a modulation that targets S-cones and attempts to silence melanopsin containing cells, the robust modulation approach reduced unwanted contrast 4-fold (FIG. 8). The robust silencing may be implemented for any biological variation that can be expressed as a spectral filter experienced by the targeted or silenced photopigment, and/or as the presence of an additional photopigment that must be silenced. The present disclosure provides examples of biological variation of these types including age, genetic variation in peak spectral sensitivity, failures to saturate rods under photopic conditions, variation in macular pigment across spatial location in the retina and hemoglobin spectral filtering through retinal blood vessels.

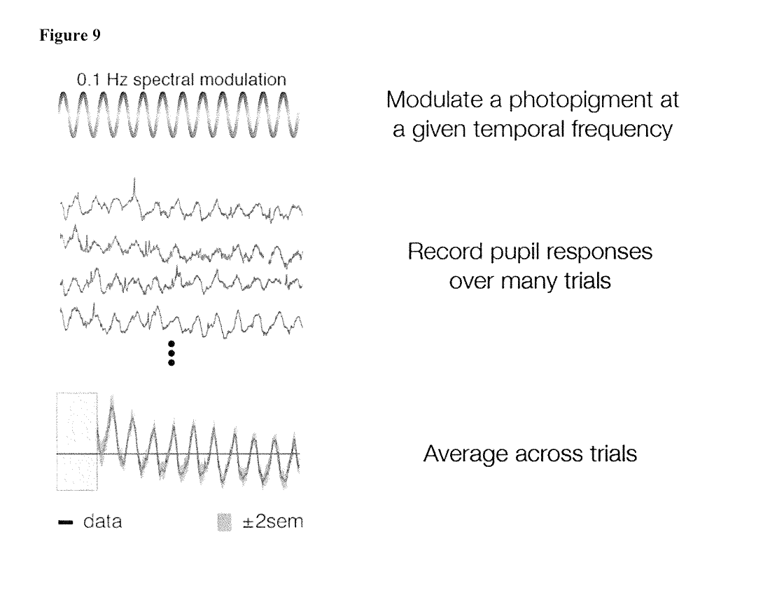

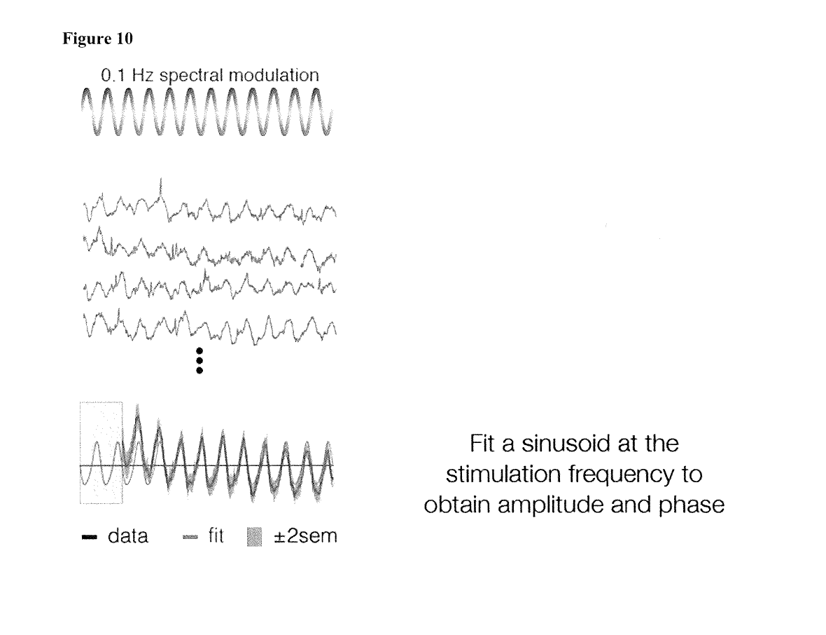

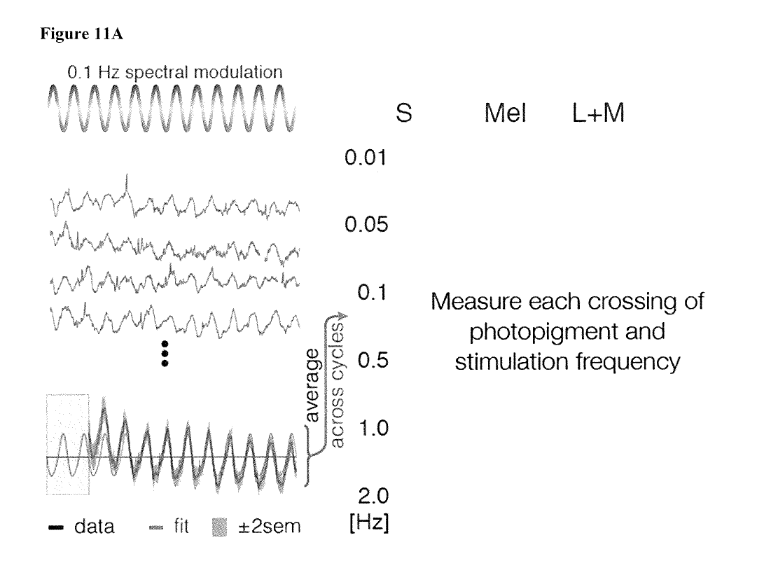

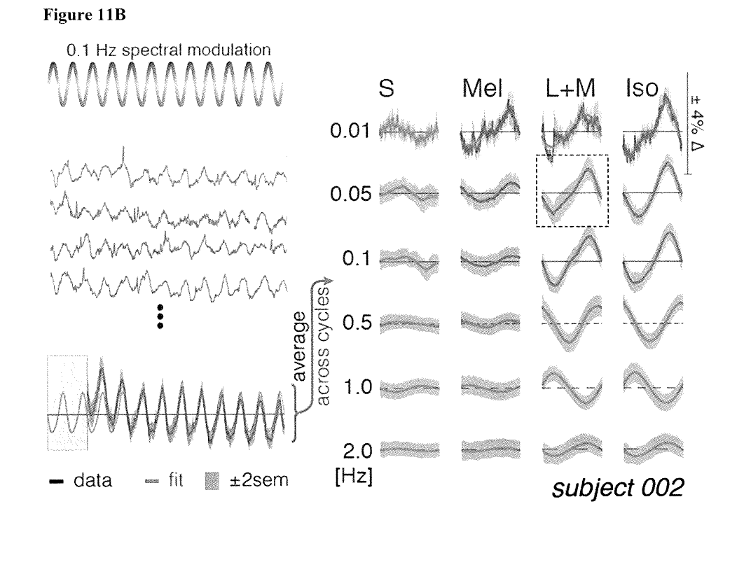

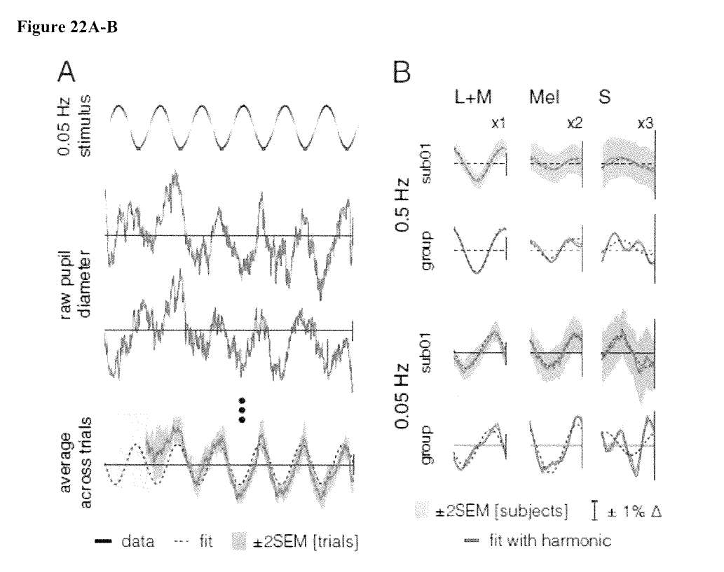

Robust spectral modulations produce measurable and distinct pupil responses to cones and melanopsin. Presentation of sinusoidal spectral modulations of different temporal frequencies (0.05, 0.1 and 0.5 Hz) and targeting different photoreceptors were presented to 16 subjects. During each period of stimulation presented to a pharmacologically dilated eye, lasting 40 or 100 seconds, the consensual pupil response was recorded using an infrared eye tracker. Averaging many such trials together of a given frequency and photoreceptor target yields an average pupil response (FIG. 9). The average response was fit with a sinusoid of the stimulation frequency to obtain the amplitude and phase of response (FIG. 10). This averaging and fitting was performed for each crossing of modulation frequency and targeted photoreceptor for each subject (FIG. 11A). Measurable pupil responses were obtained for all studied modulation frequencies and targeted photopigments (FIG. 11B). As shown in FIG. 22A, the effect of the stimulation having a temporal frequency of 0.05 Hz is apparent as a sinusoidal oscillation at the stimulation frequency in the raw traces of pupil response from one subject. The average response across cycles of the modulation at the stimulus frequency (FIG. 22B) revealed in both the individuals and the group data that the L+M- and melanopsin-directed modulations produce pupil responses of similar phase. The S-cone modulation, however, produced responses with markedly different phase.

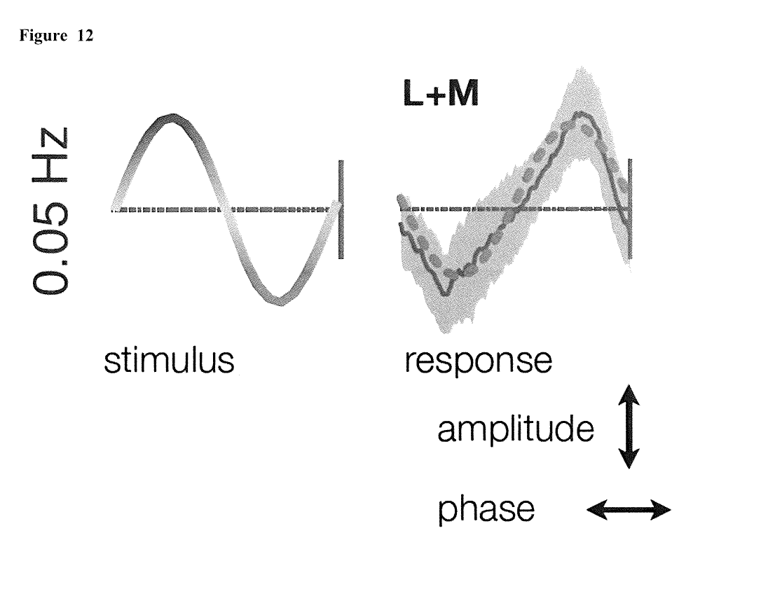

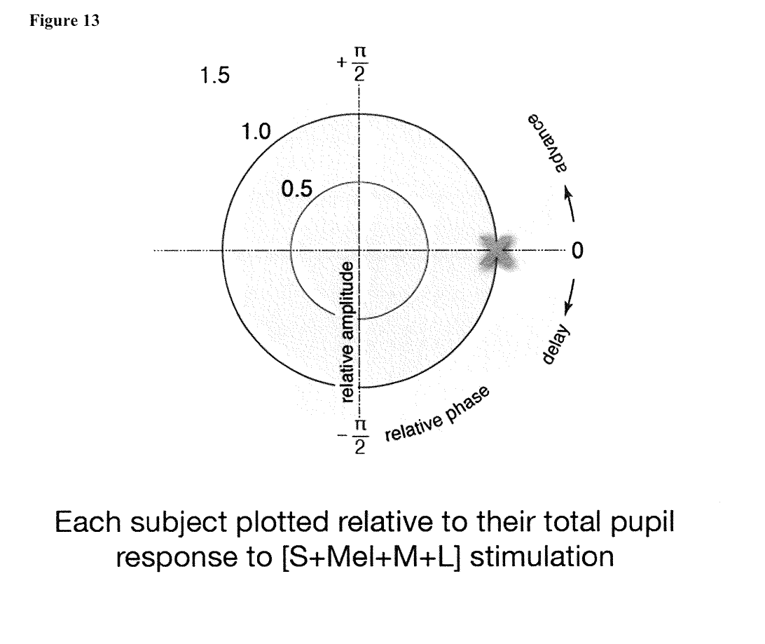

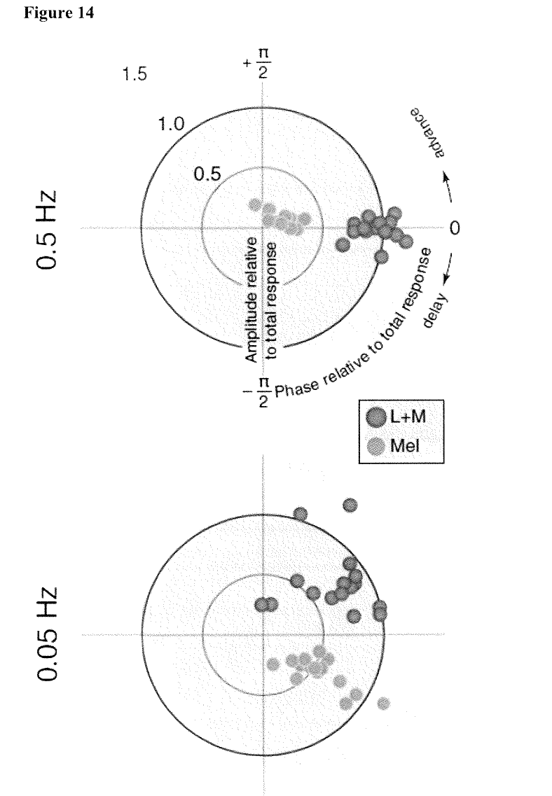

The response for each subject to each modulation frequency and targeted photopigment can be summarized by the amplitude and phase of pupil response evoked (FIG. 12). Amplitude and phase information is presented on a polar plot (FIG. 13). The relative amplitude and phase response for each subject to L+M and melanopsin modulations at the two frequencies were examined (FIG. 14). Unwanted individual differences in the amplitude and phase of response that are common to all photoreceptor driven responses (such as might be related to overall subject age or elasticity of the pupil fibers) were removed by normalizing the responses for each subject by the integrated pupil response for that subject obtained by the complex sum of the S, L+M, and melanopsin evoked responses. Normalized in this way, the pupil response data for 16 subjects for the L+M and melanopsin directed stimulation at 0.5 Hz showed consistent, measurable responses (FIG. 14). At high temporal frequency (0.5 Hz), the melanopsin- and L+M evoked pupillary responses are in phase (FIG. 14, upper). At the lower frequency (0.05 Hz), L+M- and melanopsin-evoked responses become desynchronized in quadrature phase (FIG. 14, Lower).

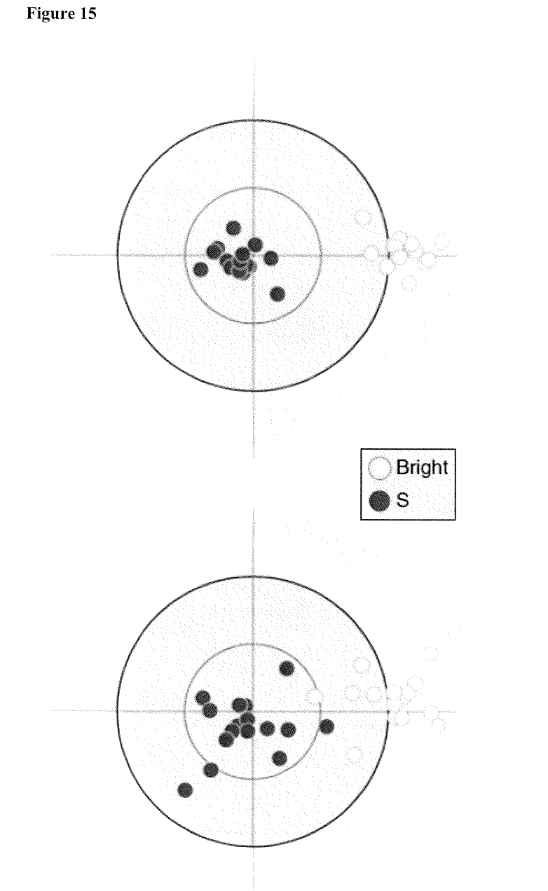

Anatomical and physiological properties of the retina suggest that L+M and Melanopsin driven pupil responses were synergistically combined into a “brightness” channel. When the sum of L+M and melanopsin responses were placed on the polar plot, robust S-cone directed stimulation was observed to produce an antagonistic pupil response (FIG. 15). Without being limited to a particular theory, the robust targeting of S-cones is critical for this finding and inference, as the S-cone directed stimulation silenced hypothetical variations in human melanopsin (the spectral sensitivity of which has been incompletely characterized) and rods that may have somehow escaped saturation under the photopic stimulation conditions and have been stimulated with out-of-phase contrast.

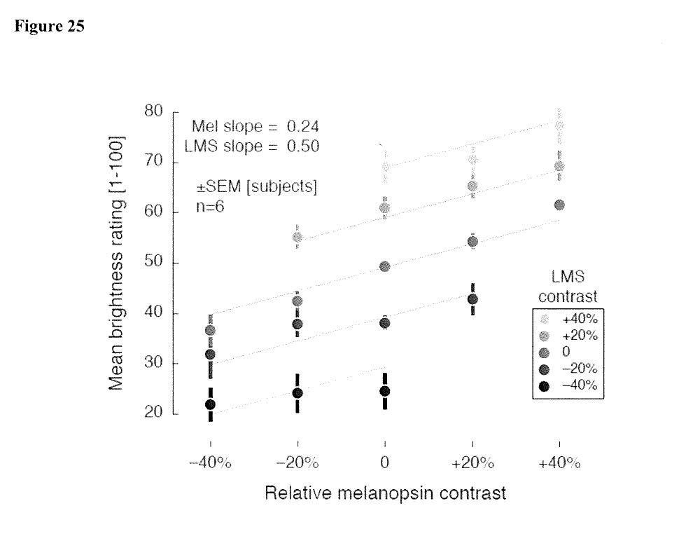

Additional experiments were performed to determine whether humans perceive melanopsin contrast as relative brightness and that this signal is additive to that from the cones. After adaptation to a background spectrum of ˜3000 cd/m², on each of many 4 second, randomized trials, six healthy control subjects rated the brightness (on a scale of 0-100) of a spatially uniform spectrum. Each spectrum produced a change in contrast on all cones (LMS) and melanopsin of ±40%, ±20%, or 0%. The five contrast levels for each photoreceptor class (LMS or melanopsin) were crossed with the other photoreceptor class (with the exception of a few crossings not available within the device gamut). FIG. 25 shows the mean brightness rating obtained across trials and subjects as a function of melanopsin contrast. Plot points were shaded depending upon the LMS contrast that was present in the stimulus. The data were fit with a model of additive, linear contributions of LMS and melanopsin contrast on brightness rating. Both LMS and melanopsin contrast were found to be highly significant contributors to brightness perception, with a roughly 2:1 ratio (respectively) of influence.

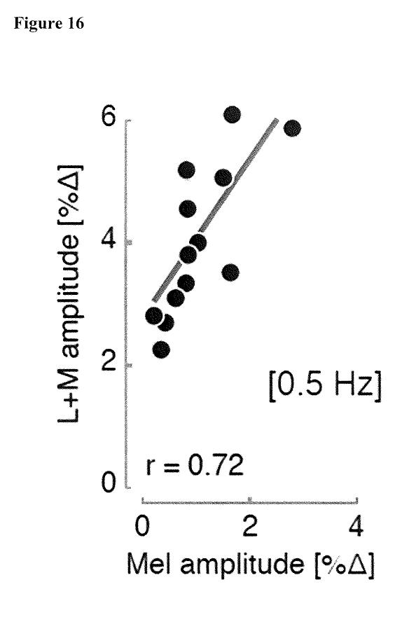

Melanopsin and L+M evoked pupil responses are correlated across subjects. In a healthy control population, the amplitude of pupil response evoked by melanopsin directed stimulation was correlated with that evoked by L+M directed stimulation (FIG. 16). Pathological conditions which relatively enhance or diminish melanopsin sensitivity can be detected as departures from this relationship seen in healthy individuals.

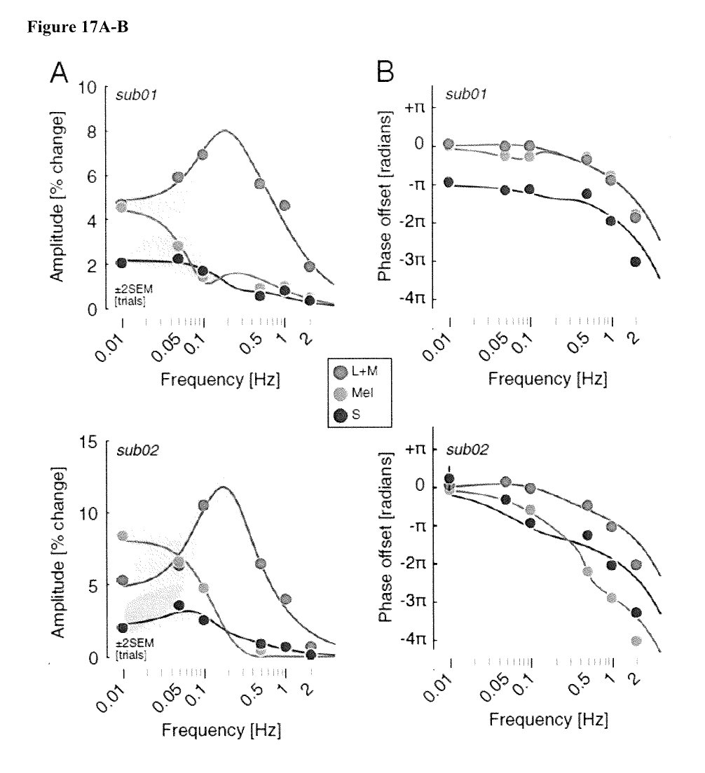

Distinct pupil temporal transfer functions are evoked by robust targeting of different photoreceptors. Despite the generally antiphase relationship of S-cone responses across subjects, there were individual differences in the phase effects greater than individual measurement error. Two subjects were analyzed in greater depth and pupil responses to the photoreceptor-directed modulations at six temporal frequencies between 0.01 and 2 Hz were measured. FIG. 17A presents the amplitude responses across temporal frequencies for both subjects. These transfer functions characterize the temporal filtering properties of each photoreceptor channel. The pupil response mediated by the L+M cone pathway was band-pass, with a maximum response at 0.1 Hz, rolling off for higher frequencies. The responses to melanopsin and S-cone stimulation were low-pass, maximal at the lowest measured modulation frequency (0.01 Hz), and markedly reduced by 0.5 Hz. In the two subjects, the pattern of amplitudes of pupil response evoked across modulation frequencies was observed to be distinct for the stimuli that targeted different photoreceptor classes (FIG. 17A), confinning the selective targeting.

FIG. 17B presents the phase of the response across frequencies of stimulation. The amplitude and phase data were modeled simultaneously with a time-invariant, linear model composed of a “fast” and “slow” temporal filter. The amplitude and phase data were well fit by the model, including a fixed temporal delay in both observers across photoreceptor channels of ˜250 ms and a negative amplitude response to S-cone modulation for the fast filter. The separate filter properties for L+M and melanopsin account for the quadrature phase desynchronization of these responses at lower temporal frequencies. Further, differences in model parameters account for the finding in subject 02 of an S-cone response that seems in-phase with L+M and melanopsin responses at low frequencies, despite having an S-cone opponent input to the fast filter of the model pupillary light response. This individual difference arises, at least in part, because the slow filter S-cone component is of the same sign as the melanopsin component of the response for this subject.

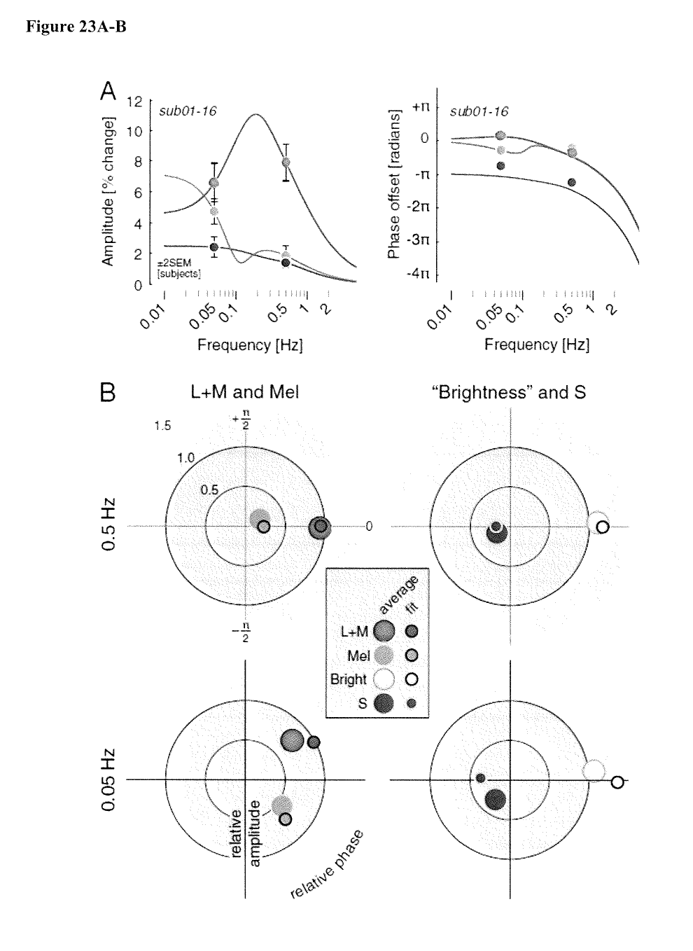

This model was then applied to the group data. The average amplitude and phase of response across the 16 subjects for each combination of photoreceptor target and modulation frequency. The two-filter model fits the average amplitude and phase data (FIG. 23A) with parameters similar to those found for subject 01. When expressed as a polar plot (FIG. 23B), the agreement between the group data and model fits is apparent. Interestingly, there is systematic “rotation” of the phase of both the pupil brightness and S-cone responses at the lower temporal frequency that is not captured by the model. This may result from individual differences in the phase of S-cone responses at low temporal frequencies, as is seen between subject 01 and subject 02 (FIG. 17), because the average data do not fully constrain the model and the fits shown are based on parameters obtained for subject 01.

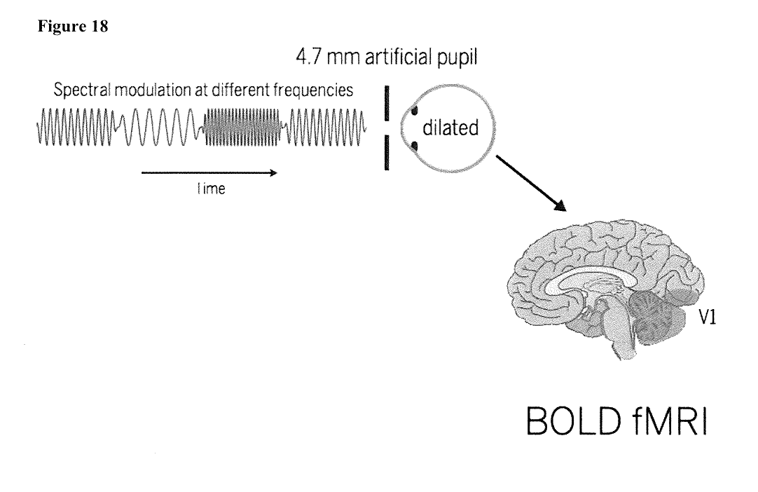

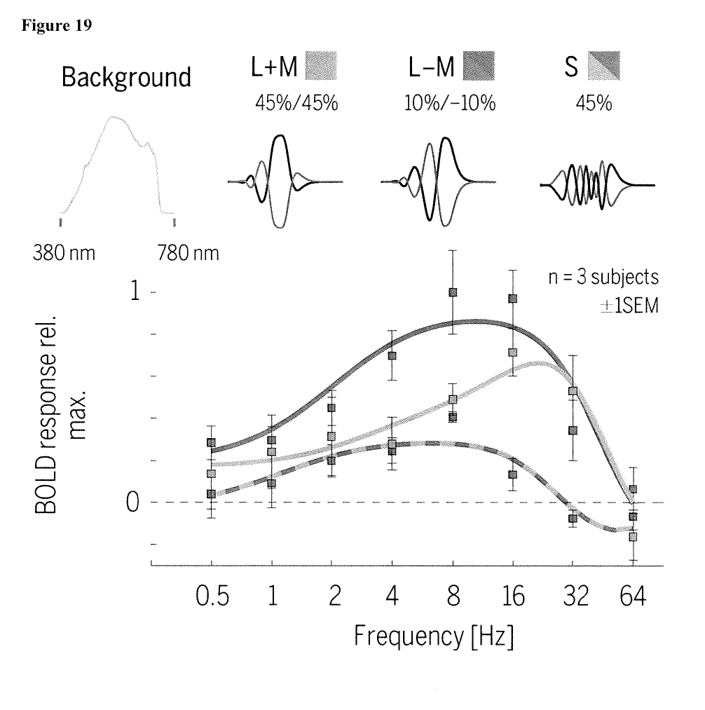

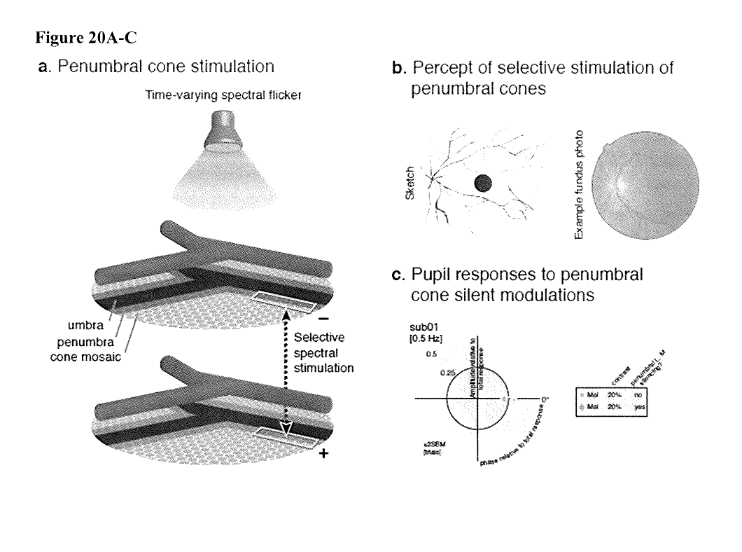

Distinct neural temporal transfer functions to flicker are evoked by robust targeting of different photoreceptors. Periods of photoreceptor directed flickering light were presented to three subjects while they underwent Blood Oxygen Level Dependent (BOLD) fMRI brain scanning (FIG. 18). The modulations included L+M, L−M (stimulating the red-green opponent chromatic channel) and S. Modulations were presented at a range of log spaced flicker frequencies between 0.5 and 64 Hz. Whole field, photoreceptor directed flicker evoked measurable neural response from the primary cortical visual area of the brain (FIG. 19). Distinct flicker temporal transfer functions for different targeted photoreceptors and their combinations were observed. Flicker directed at L+M and L−M cone pathways evoked robust responses, peaking at 8-16 Hz (FIG. 19). Rapid melanopsin stimulation produced responses that could not be distinguished from the noise properties of the measurement.

### Example 2

### Selective Targeting of Penumbral Cones

Retinal blood vessels are located between incoming light and the photosensitive cells of the eye (FIG. 20A). Light passing through the hemoglobin present in the blood undergoes spectral filtering. Some cells completely in the shadow (umbra) of blood vessels receive minimal light stimulation and are never exposed to unfiltered light. There is evidence that signals from these cells are not represented in the brain. Other cells (in the penumbra) are partially shaded and do produce physiologically relevant signals. The vast majority of photosensitive cells are unshaded and lie in the open field of the retina.

A spectral modulation of light designed to produce isolated stimulation (for example) of melanopsin and silence L and M cones can succeed in silencing L and M cones in the open field of the retina, yet produce inadvertent stimulation of L and M cones within the shadow (penumbra) of blood vessels. This is because a spectral modulation that is well tuned to silence open field L and M cones can undergo spectral filtering by blood vessels and the resulting, modified spectra can produce contrast on the penumbral cones.

The robust selective stimulation of the present disclosure can be used either to selectively target penumbral cones, or to silence them. As a demonstration of the power of the robust isolating method, a spectral modulation was created that silenced melanopsin, S, L and M cones but produced approximately 2% predicted contrast on penumbral L and M cones. When flickered at 16 Hz, viewing of this modulation produced a distinct percept of the “Purkinje tree”, which is the pattern of blood vessels on the surface of the retina. A naive observer was asked to view the modulation and sketch her visual impression (FIG. 20B). The resulting sketch demonstrates the normal vascular anatomy of the retina, and is readily comparable to an example photograph of the retinal blood vessels seen on the fundus of the eye.

Stimulation of the penumbral cones can be undesirable if, for example, the aim is to measure the response to isolated melanopsin stimulation. FIG. 20C shows the amplitude and phase of the pupil response measured to a modulation directed at melanopsin that either does or does not silence the penumbral cones using the disclosed robust isolation technique. As can be seen, a similar amplitude and phase of pupil response was recorded under these conditions (which is to be expected given the predicted small contribution of penumbral cones to pupil response). This demonstrates that physiologic responses to penumbral-cone silent modulations can be recorded.

### Example 3

### Pupillometric Assessment of High Temporal Frequency Visual Function

This Example describes a method and application of pupillometry that is sensitive to visual function at high temporal frequencies. This approach implements an amplitude modulation of photopigment directed spectral contrast that reveals a non-linearity in visual function, resulting in a measurable pupil response.

The pupil response is an easily obtained, objective, quantifiable measurement of the function of the visual system. A limitation of pupillometry for assessment of visual function is that movement of the pupil is slow relative to the response properties of the retinal cells. Retinal cells (and human visual perception) are sensitive to temporal frequencies as high as 50 Hz (50 cycles per second), but the mechanical action of the pupil is limited to approximately 5 Hz. Consequently, disorders of retinal function that first manifest as a loss of high temporal frequency function are not detectable with traditional pupillometric methods. An application of robust photoreceptor targeting is to create amplitude-modulated stimuli that evoke a non-linearity in the pupil response to probe visual function at high temporal frequencies.

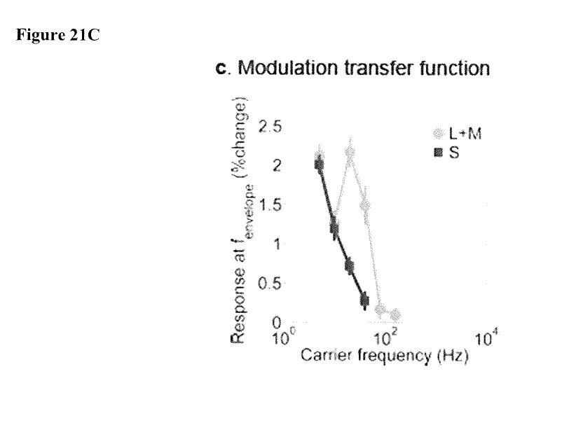

In this application, the device produces an amplitude-modulated flicker of light (FIG. 21A). The modulation has both a lower envelope frequency (e.g., 0.5 Hz) and a higher carrier frequency (ranging from 5 to 50 Hz). If the response properties of the visual system were strictly linear, then this modulation would produce no net pupil response, as the brightness of the light is, on average, constant within the temporal range of pupil responsiveness. Instead, a measurable pupil response was found at the envelope frequency under these conditions (FIG. 21B). This non-linear response is termed the distortion product (see Stockman and Plummer, Vision Research, 38(23), 3703-3728, 1998). The amplitude of the distortion product at different carrier frequencies can be measured, producing a photoreceptor specific measure of the modulation transfer function (FIG. 21C).

The amplitude of the pupil response at different carrier frequencies can be measured, producing a measure of the modulation transfer function at temporal frequencies higher than the pupil can respond to directly. The key idea here is that low-frequency modulation of carrier allows low frequency pupillary readout of the response of the visual system to the higher-temporal frequency modulation of the carrier.

An important refinement of the approach is that, instead of a broad spectrum flicker of light, tailored spectral modulations can be used that produce relatively selective stimulation of particular photopigment systems. In addition, the use of envelope and carrier frequencies can be used to target different cone classes, e.g., the carrier frequency can stimulate the L and M cones selectively, while the envelope frequency can stimulate melanopsin selectively. When coupled with the amplitude modulation approach, the temporal response properties of the different photopigment systems can be assessed individually.

### Example 4

### Robust Targeting Across Retinal Location

The pigmentation of the human eye varies by retinal location. The macula, located around the center of fixation, contains pigments that absorb blue wavelength light. Photoreceptors in this region of the retina receive a spectrum of light that has been filtered differently from that at other retinal locations. As a consequence, a spectral modulation designed to stimulate melanopsin and not L or M cones can have the desired specificity in the periphery of the retina, but would have undesired contrast on L and M cones in the area of the macula. Traditionally, this problem has been approached by requiring a subject to maintain fixation while stimulation is isolated, e.g., to the periphery of the retina. This requirement to control the location of stimulation across the retina to achieve photoreceptor targeting limits clinical application, as fixation in general is difficult for people and is specifically not possible for patients with a loss of central vision or nystagmus (an uncontrolled movement of the eyes).

An application of robust photoreceptor targeting is to render the stimulation relatively insensitive to location across the retina. The modulation is tailored to silence classes of photoreceptors across the retina, accounting for the difference in spectral filtering that they receive as a consequence of retinal location. This allows for free viewing of a full-field stimulus while retaining control of selective photoreceptor targeting.

### Example 5

### Square Wave Spectral Modulations Separately Target Photoreceptor Classes

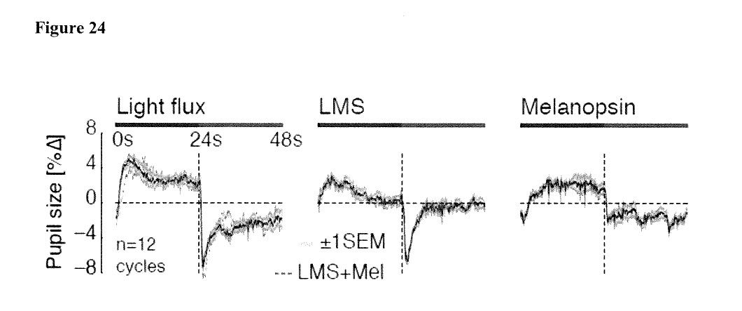

This Example shows that square wave spectral modulations (as opposed to sinusoidal) also generate separable profiles of response in the pupil. FIG. 24 shows the pupil response from a subject viewing slow (48 seconds per cycle) square-wave, isochromatic spectral modulations. Each plot shows the average (across cycles) pupil response to a transition from the negative (black; right panel) to the positive (dark gray; left panel) state of the modulation. The responses to all the cones (LMS) and melanopsin stimulation have distinct time courses, and the sum of the LMS and melanopsin responses (dashed line) matches the response to the light flux modulation. Notable is the sustained response to melanopsin, which is absent in the cone-driven response.

### Example 6

### Melanopsin, but not Cone, Stimulation Provokes Squinting

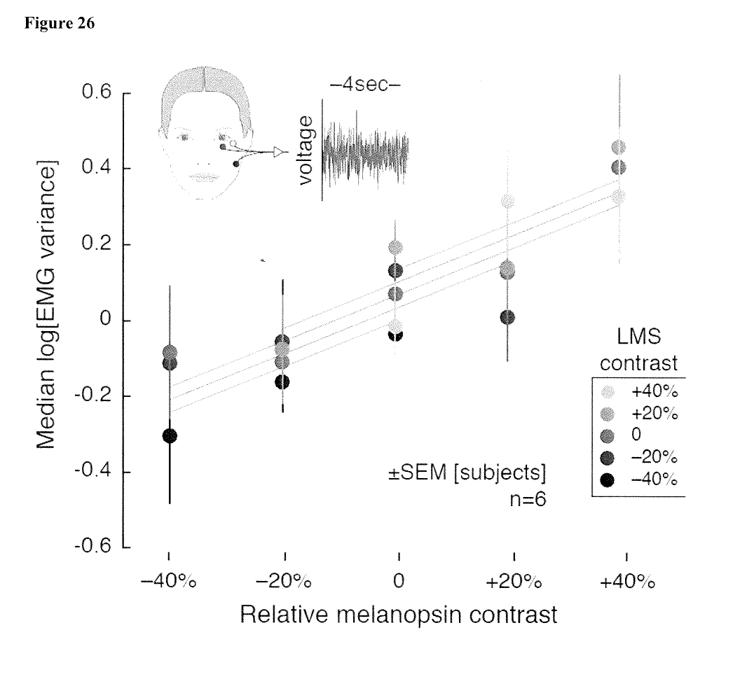

During performance of the brightness rating task (FIG. 25), orbicularis oculi electromyography (EMG) was simultaneously recorded from surface electrodes positioned below the lower lid of the stimulated eye (Nahar 2007). The variance of the EMG signal during the measurement period was driven by eyelid squint, which in turn reflects visual discomfort as induced by glare or refractive error (Stringham 2004, Nahar 2007, Gowrisankaran 2007). Subjects were told only that the electrodes measure “the eye muscles” and were naive to the hypothesis that bright lights might cause squinting. As shown in FIG. 26, there is a significant increase in squint associated with increasing melanopsin contrast. The absolute size of this change is small; effectively a slight narrowing of the eyes with greater melanopsin stimulation. Interestingly, cone contrast, even though perceived as a greater source of reported brightness, had no effect upon squint. These data demonstrate the usefulness of EMG as a measure in concert with robust photoreceptor selective stimulation.

All patents, patent applications, publications, product descriptions and protocols, cited in this specification are hereby incorporated by reference in their entirety.

While it will be apparent that the disclosure herein described is well calculated to achieve the benefits and advantages set forth above, the present disclosure is not to be limited in scope by the specific embodiments described herein. It will be appreciated that the disclosure is susceptible to modification, variation and change without departing from the spirit thereof.

What is claimed is:

1. A system comprising:
(a) a digital spectral integrator;
(b) a detection means; and
(c) an integration means,
wherein the digital spectral integrator produces a light of a specified spectral and temporal profile under the control of the integration means, and the detection means detects a response to the light of a specified spectral and temporal profile.

2. The system of claim 1, wherein the detection means is an infrared camera, electroretinogram, electroencephalogram, electromyogram, functional MRI scanner or recording of a subject's behavioral responses.

3. The system of claim 1, wherein the integration means is a software program.

4. The system of claim 3, wherein the software program interprets the response to the light of a specified spectral and temporal profile.

5. The system of claim 1, wherein the light of a specified spectral and temporal profile comprises a carrier frequency.

6. The system of claim 5, wherein the carrier frequency is about 0.01 Hz to about 128 Hz.

7. The system of claim 1, wherein the light of a specified spectral and temporal profile comprises a lower envelope frequency and a higher carrier frequency.

8. The system of claim 7, wherein the higher carrier frequency is about 4 to about 128 Hz.

9. The system of claim 1, wherein the response is pupil movement, electrical response of the retina, neural activity of the brain or perceptual report of the subject.

10. The system of claim 1, wherein the light of a specified spectral and temporal profile is provided as a spatially uniform field of light.

11. A method of detecting a response or producing a biological effect associated with selectively stimulating a photosensitive molecule comprising:
(a) contacting the photosensitive molecule with a light of a specified spectral and temporal profile;
(b) robustly silencing responses from non-targeted photosensitive molecules despite biological variation in the effective spectral sensitivities of the photosensitive molecules; and
(c) detecting the response or biological effect associated with selective stimulation of the photosensitive molecule.

12. The method of claim 11, wherein the photosensitive molecule has been artificially introduced into neural tissue.

13. The method of claim 11, wherein the photosensitive molecule is a photosensitive protein.

14. The method of claim 13, wherein the photosensitive protein is present in a photosensitive cell.

15. The method of claim 14, wherein the photosensitive cell is a retinal cell.

16. The method of claim 15, wherein the retinal cell is a rod, cone or a melanopsin-expressing retinal ganglion cell.

17. A method of detecting an impairment in a temporal sensitivity of a photosensitive cell in a subject comprising:
(a) contacting the photosensitive cell with a light of a specified spectral and temporal profile to selectively stimulate a photosensitive molecule; and
(b) detecting the response associated with selective stimulation of the photosensitive molecule to determine the temporal sensitivity of the photosensitive cell,
wherein detection of an impairment in the temporal sensitivity of the photosensitive cell is indicative that the subject has an ophthalmologic, neurological or psychiatric disorder.

18. The method of claim 17, wherein the photosensitive cell is a retinal cell.

19. The method of claim 18, wherein the retinal cell is a rod, cone or a melanopsin-expressing retinal ganglion cell.

20. The method of claim 17, wherein:
(a) the ophthalmologic disorder is selected from the group consisting of retinitis pigmentosa, glaucoma, macular degeneration, Leber's Congenital Amaurosis and diabetic retinopathy;
(b) the neurological disorder is selected from the group consisting of migraines, photophobia, traumatic brain injury, neurodegenerative disorders involving the brainstem and Progressive Supra-nuclear Palsy; or
(c) the psychiatric disorder is selected from the group consisting of seasonal affective disorder and mood disorders.
